(19) United States
(12) Patent Application Publication
(10) Pub. No.: US 2023/0381778 A1
(43) Pub. Date: Nov. 30, 2023

(54) DEVICES, SYSTEMS, AND METHODS RELATED TO NUCLEIC ACID ISOLATION

(71) Applicant: University of Florida Research Foundation, Inc., Gainesville, FL (US)

(72) Inventors: Benjamin Earl VALLEY, Gainesville, FL (US); Jason E BUTLER, Gainesville, FL (US); Anthony J. LADD, Gainesville, FL (US)

(21) Appl. No.: 18/245,947

(22) PCT Filed: Sep. 24, 2021
(86) PCT No.: PCT/US2021/051885

§ 371 Date: Mar. 20, 2023

Related U.S. Application Data
(60) Provisional application No. 63/083,244, filed on Sep. 25, 2020.

(51) Int. Cl. B01L 3/00

(52) U.S. Cl. CPC B01L 3/50273; B01L 3/502715; B01L 2300/16; B01L 2400/0421

(57) ABSTRACT

Provided herein are microfluidic devices that can be configured to generate an electrophoretic flow that is in opposition to a fluid flow through a microcapillary of a microfluidic device provided herein for nucleic acid isolation. Also provided herein are systems comprising such. Also provided herein are methods that include adding an amount of a sample comprising nucleic acids to the inlet area of a microfluidic device as provided herein, generating a first fluid flow through a microcapillary of a microfluidic device provided herein; and applying a uniform electric field to the microfluidic device, where the uniform electric field generates an electrophoretic flow that is in opposition to the fluid flow. Methods as described herein can further comprise quantifying or otherwise characterizing isolated nucleic acids separated by devices, systems, and methods as described herein.

### CROSS-REFERENCE TO RELATED APPLICATIONS

This application claims the benefit of and priority to U.S. Provisional Patent Application No. 63/083,244 entitled “DEVICES, SYSTEMS, AND METHODS RELATED TO NUCLEIC ACID ISOLATION” filed on Sep. 25, 2020, which is expressly incorporated by reference as if fully set forth herein in its entirety.

### STATEMENT REGARDING FEDERALLY SPONSORED RESEARCH OR DEVELOPMENT

This invention was made with government support under Grant No. 1804302, awarded by the National Science Foundation. The government has certain rights in the invention.

### BACKGROUND

Separation and concentration of nucleic acids and polynucleotides can play significant roles in many research, diagnostic, forensic, and clinical applications. As such, there exists a need for improved devices and techniques for separating and/or concentrating nucleic acids and polynucleotides, in particular ribonucleic acid (RNA) from compositions comprising polynucleotides.

### SUMMARY

Described herein are devices, systems, and methods for the separation and/or isolation of nucleic acids from a sample.

In embodiments, described herein are microfluidic devices. In an embodiment, a microfluidic device for isolating or detecting nucleic acids comprises a microcapillary having a first end and a second end with a length (L) longer than a width (W), and wherein one or more inner surfaces of the microcapillary are coated with a coating; a fluid inlet at the first end fluidically connected to the microcapillary; and a fluid outlet at the second end fluidically connected to the microcapillary. In embodiments, the first end, the second end, or both are greater in cross-sectional area than the section of microcapillary between the first and second ends. In embodiments, the width of the microcapillary ranges from about 0.1 μm to about 2 mm. In embodiments, the length of the microcapillary ranges from about 100 μm to about 1 m. In embodiments, the cross-sectional area of the microcapillary is trapezoidal in geometry. In embodiments, the coating of the microcapillary alters osmotic flow. In embodiments, the coating of the microcapillary enhances osmotic flow. In embodiments, the coating is PDMAC or PDADMAC. In embodiments, the microfluidic device comprises one or more of polymethylmethacrylate (PMMA), polydimethylsiloxane (PDMS), polycarbonate, polystyrene, polyethylene, or glass. In embodiments, the microfluidic device comprises fused polymethylmethacrylate sheets. In embodiments, devices as described herein further comprise a sample injection port fluidically connected to the microcapillary and positioned in between the fluid inlet and fluid outlet on the first end of the microcapillary.

Described herein are systems for separating, isolating, and/or otherwise detecting nucleic acids. In embodiments, a system for isolating or detecting nucleic acids, comprises a microfluidic device as described herein and a buffer. In embodiments of systems as described herein, the buffer has an ionic concentration of about 0 mM to about 10 mM. In embodiments of systems as described herein, the buffer has a pH of about 6.5 to about 8.5. In embodiments of systems as described herein, systems further comprise an electric current generator configured to generate an electrophoretic flow through the microcapillary. In embodiments of systems according to the present disclosure, systems can comprise an inlet tank fluidically connected to the fluid inlet and an outlet tank fluidically connected to the fluid outlet. In embodiments of systems as described herein, system further comprise a valve in fluidic communication between the inlet tank and outlet tank. In embodiments of systems as described herein, systems can further comprise a syringe pump in fluidic communication with the fluid outlet. In embodiments of systems as described herein, systems can further comprise a valve in fluidic communication with the inlet tank and fluid inlet, upstream of the fluid inlet. In embodiments of systems as described herein, systems can further comprise a valve in fluidic communication with the outlet tank and fluid outlet, downstream of the fluid outlet. In embodiments of systems as described herein, systems can further comprise a pump to drive fluid motion through the system. In embodiments of systems as described herein, the microfluidic device, inlet tank, and outlet tank form a closed loop. In embodiments of systems as described herein, systems can further comprise a translational stage operably connected to the outlet tank. In embodiments of systems as described herein, systems can further comprise a computing controller. In embodiments of systems as described herein, systems can further comprise a micrograph image collecting apparatus. In embodiments of systems as described herein, the coating of the microcapillary suppresses osmotic flow. In embodiments of systems as described herein, the coating is a charge-neutral polymer.

Further described herein are methods for isolating and/or detecting and/or otherwise separating nucleic acids. In an embodiment, a method for isolating or detecting nucleic acids, comprises providing a microfluidic device or a system as described herein; providing a sample comprising nucleic acids to the sample inlet or sample injection port of the microcapillary; generating a fluid flow from the fluid inlet to the fluid outlet with a centerline velocity vₒ along a longitudinal axis of the microcapillary; and providing an electric current to the microcapillary, wherein the electric current is configured to generate an electrophoretic velocity vₑ that is directionally opposed to the fluid flow and centerline velocity vₒ; and detecting or isolating nucleic acids at a stagnation region of interest near the fluid inlet. In embodiments of methods as described herein, methods further comprise illuminating the microfluidic device with light from a light source for a period of time prior to providing the sample. In embodiments of methods as described herein, the light source is a mercury lamp. In embodiments of methods as described herein, the period of time is about two hours. In embodiments of methods as described herein, the ratio of centerline velocity vₒ to electrophoretic velocity vₑ is about 20 to about 200. In embodiments of methods as described herein, the centerline velocity vₒ is about 4 mm/s to about 12 mm/s. In embodiments of methods as described herein, the electrophoretic velocity vₑ is about −0.03 mm/s to about −0.15 mm/s or about +0.03 mm/s to about +0.15 mm/s. In embodiments of methods as described herein, the nucleic acids comprise nucleic acids with a λ_{D} greater than the diameter of the backbone. In embodiments of methods as described herein, the diameter of the backbone is about 2 nm. In embodiments of methods as described herein, the nucleic acids comprise at least one nucleic acid with a length of at least 30 to 100 bases with a sequence having at least 95% sequence homology or greater with a viral RNA sequence of SARS-CoV-2. In embodiments of methods as described herein, the nucleic acids comprise at least one nucleic acid with a length of at least 30 to 100 bases with a sequence having at least 95% sequence homology or greater with a viral RNA sequence of NCBI Reference Sequence NC_045512.2. In embodiments of methods as described herein, detecting or isolating nucleic acids at a stagnation region of interest near the fluid inlet comprises isolating DNA and RNA and detecting only RNA or only DNA.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is an embodiment of a device as described herein.

FIGS. 2A-2B show embodiments of a microfluidic trap where an inlet area and/or an outlet area are part of a microcapillary.

FIGS. 3A-3B show embodiments of a microfluidic trap that does not comprise an outlet area.

FIG. 4 shows an embodiment of a microfluidic trap having multiple inlet areas and outlet areas.

FIG. 5 shows an embodiment of a microfluidic trap configured to collect concentrated particles via a fluid flow that is not parallel to the fluid flow used to drive sample particles into the microcapillary.

FIGS. 6A-6C: (FIG. 6A) is a cartoon illustration of the nucleic acid isolation mechanism. DNA molecules (green) enter the contraction channel due to the large convective flow (blue). The electric field induces an opposing electrophoretic velocity (orange) that is less than 10% of the mean convective velocity. DNA molecules migrate toward the channel walls in response to the simultaneous application of the two fields as indicated by the blue-orange arrows. DNA molecules near the wall return to the entrance of the channel contraction (orange arrows) because the electrophoretic velocity near the wall exceeds the fluid velocity. Upon reaching the expansion DNA molecules accumulate in a thin layer near the bounding walls. FIG. 6B is a brightfield micrograph of an embodiment of the nucleic acid isolation device (only a portion of the length of the device is shown). The three circles, from left to right are for buffer inlet, sample injection, and buffer outlet ports. The red solid and dashed squares mark where measurements were taken. FIG. 6C is a schematic of an embodiment of the experimental setup.

FIGS. 7A-7B illustrate DNA accumulation kinetics according to systems and methods described herein with 10 μL samples containing 0.1 ng/μL DNA and 30 mg/mL BSA. (FIG. 7A) Fluorescent micrographs of DNA at the contraction entrance after 15 minutes and (FIG. 7B) Time-dependent average DNA concentrations measured at the contraction entrance. The centerline fluid velocities, v₀, and opposing electrophoretic velocities, vₑ, of (i)-(iv) in FIG. 7A were respectively, (i) 4.4 and 0.09 mm/s (red stars), (ii) 4.4 and 0.12 mm/s (red squares), (iii) 8.8 and 0.12 mm/s (closed blue circles), and (iv) 11 and 0.15 mm/s (purple triangles). Open blue circles are for BSA free sample with v₀=8.8 mm/s and vₑ=0.12 mm/s. Error bars indicate one standard deviation (n=3).

FIGS. 8A-8B are graphs depicting BSA elution rates with 10 μL samples containing 0.1 ng/μL DNA and 30 mg/mL BSA (2.5% wt. FITC-BSA) measured at the contraction channel entrance of an embodiment of a device as described herein. The following combinations of v₀ and vₑ were used: 4.4 and 0.12 mm/s (red squares), 8.8 and 0.12 mm/s (blue circles), salted samples (open blue circles), and 11 and 0.15 mm/s (purple triangles). (FIG. 8A) High concentration BSA eluting past the detector, measured using 8× neutral density filtering. (FIG. 8B) Trace BSA concentrations eluting at longer times, measured without neutral density filtering. Error bars indicate one standard deviation (n=3).

FIGS. 9A-9B are graphs plotting total mass of DNA trapped in device for DNA/BSA mixture samples containing 1 ng DNA and 0.3 mg BSA. (FIG. 9A) Example of concentrations at the outlet (indicated by the dashed red square in FIG. 6B) calculated from each image following the release of the trap (by setting E=0) at t=0. Respective parameter combinations of v₀ and vₑ used are 4.4 and 0.12 mm/s (red squares), 8.8 and 0.12 mm/s(blue circles), and 11 and 0.15 mm/s (purple triangles). (FIG. 9B) Average calculated DNA masses trapped in the device under different parameter combinations for processing times of 20 minutes (orange bars), 20 minutes with salted samples (blue bars), and 40 minutes (red bars). Error bars indicate one standard deviation (n=3).

FIGS. 10A-10E are photographs of an embodiment of a reduced-to-practice embodiment of an acrylic microfluidic device as described herein. Digital images of (FIG. 10A) the assembled device and (FIG. 100) the device during sample injection by a user. (FIG. 10B) Scanning electron micrograph (SEM) of a cross section of the contraction channel. Brightfield micrographs of (FIG. 10D) the contraction channel entrance and (FIG. 10E) the exit region of the contraction channel corresponding to solid and dashed red squares in FIG. 6B, respectively (acquired using Nikon Diaphot 200 microscope described in the experimental section).

FIGS. 11A-11B are plots of velocity calibrations. (FIG. 11A) Average centerline fluid velocity (v₀) exhibits a linear relationship with reservoir height difference (n=3). (FIG. 11B) Average electrophoretic velocities (vₑ) of DNA (black squares; n=3) and BSA (open black circles; n=1) show linear relationships with applied voltage. The solid and dashed red lines are linear fits of the data. The error bars indicate one standard deviation.

FIGS. 12A-12B are plots of average baseline pixel intensity ( [symbol] I₀ [symbol] ) vs. Photobleaching time (green and blue open circles) and recovery time (green and blue circles) of the PMMA device's autofluorescence signal using (FIG. 12A) green and (FIG. 12B) blue excitation filters. Images were taken at the channel entrance (red square in FIG. 6B).

FIGS. 13A-13B are plots of example DNA concentration calibrations from relationship between average baseline-corrected pixel intensity, [symbol] I* [symbol] , to known sample concentration of DNA (ng/μL). (FIG. 13A) [symbol] I* [symbol] )vs. DNA concentration at the contraction channel entrance (solid red square in FIG. 6B). Data acquired using 8× neutral density filter and gain setting of 30. (FIG. 13B) [symbol] I* [symbol] )vs. DNA concentration near the contraction channel exit (dashed red square in FIG. 6B). Data acquired without neutral density filtering and a gain setting of 39. The solid red lines are linear fits of the data. The error bars indicate one standard deviation (n=2).

FIGS. 14A-14B are plots of example BSA concentration calibrations from the relationship between average baseline-corrected pixel intensity, [symbol] I* [symbol] , to concentration of BSA (mg/mL) at the contraction channel entrance (solid red square in FIG. 6B). (FIG. 14A) Data acquired with 8× neutral density filter. (FIG. 14B) Data acquired without neutral density filtering. The solid red lines are linear fits of the data. The error bars indicated one standard deviation (n=2).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry and the like, which are within the skill of the art. Such techniques are explained fully in the literature.

### Definitions

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA.

As used herein, “polynucleotide” refers to an oligomer and polymers of nucleotides.

As used herein, “particle” refers to any object that is anisotropic in either shape or charge distribution, including particles, nanoparticles, soft spherical and non-spherical objects, and molecules, that are electrophoretic and/or deformable. Particles can include, without limitation, nucleic acids (e.g. DNA and RNA), proteins, and carbon nanotubes.

As used herein, “inlet area” refers to the three dimensional area adjacent to and/or surrounding the inlet to the microfluidic channel. The inlet area can be part or all of a reservoir that can comprise a fluid. The inlet area can be part of a microcapillary. The inlet area can be part of any other container, capillary, microcapillary, tube, pipeline that can feed into the microcapillary.

As used herein, “outlet area” refers to the three dimensional area adjacent to and/or surrounding the inlet to the microfluidic channel. The outlet area can be part or all of a reservoir that can comprise a fluid. The outlet area can be part of a microcapillary. The outlet area can be part of any other container, capillary, microcapillary, tube, pipeline that can receive fluid flow from the microcapillary.

As used herein, “capture molecule” refers to a molecule that is configured to specifically bind one or more biomarker molecules of interest. A capture molecule can be a nucleotides, antibody, antigen, apatmer, affibody, polypeptides, peptides, or combinations thereof that specifically bind one or more biomarkers of interest.

As used herein “attached” as applied to capture molecules of an array refers to a covalent interaction or bond between a molecule on the surface of the support and the capture molecule so as to immobilize the capture molecule on the surface of the support.

As used herein “operatively-linked” as applied to capture molecules of an array refers to a non-covalent interaction between the surface of the support and the capture molecule so as to immobilize the capture molecule on the surface of the support. Such non-covalent interactions include by are not limited to, entrapment by the surface substrate, ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used herein, “multilayered microfluidic device” refers to a microfluidic device that comprises more than one individual microfluidic devices, where the individual microfluidic devices can be fluidly coupled and/or physically coupled to one or more other microfluidic devices of the multilayered microfluidic device and where the individual microfluidic devices are arranged within the multilayered microfluidic device such that there is more than one level of microfluidic devices in any given dimension.

### Discussion

Described herein are systems, devices, and methods relating to the purifying, concentrating, and fractionating nucleic acids (NAs) in a custom microfluidic device. Systems, devices, and methods as described herein can be useful, for example, for NA sample preparation from clinical specimens to be used in downstream analysis or for detection of NAs of specific lengths such as long RNA viruses.

Systems and devices as described herein can comprise one or more microfluidic devices. In embodiments according to the present disclosure, systems and devices as described herein can be fabricated from acrylic sheets. In embodiments according to the present disclosure, systems and devices as described herein can be fabricated from glass. In embodiments according to the present disclosure, systems and devices as described herein can be fabricated from polydimethylsiloxane (PDMS).

Devices as described herein exhibit one or more changes in cross-sectional area across the length of the device (more specifically the length of the microcapillary of the device). The change in cross-sectional area (i.e., expansion/contraction) is required to trap and concentrate the target particles (DNA/RNA/etc.) at a specific location. Without the change in cross section, the particles can be prevented from leaving the device, but will not be trapped at a specific location.

Trapping at a particular location requires that that the particle velocities caused by the flow and electric fields either cancel each other or disappear. In the case of systems and devices as described herein, the velocities disappear. The flow velocity next to the wall of the channel is zero, hence the fact that the particles migrate to the wall is critical. However, the velocity due to the electric field is not zero near the wall, and the particle moves against the direction of fluid flow. Only after the particle reaches the entry does the velocity due to the electric field drop to zero (or at least a small value) since the electric field drops to zero (of at least a small value). The electric field is lower because of the change in the cross section size: the strength of the electric field is (roughly) inversely proportional to the cross sectional area. Consequently, we must design the cross sectional shape of the device accordingly to cause the particle to trap and concentrate.

Note that without the change in cross section, a separation can presumably be made but the particles will not be trapped or concentrated.

Such devices can comprise an expansion-contraction microchannel connected to reservoirs comprising buffer solution. Samples (microLiters) can be directly injected into the device's expansion channel with a chromatography syringe via a rubber septum. Using a pressure-driven flow, together with an electric field that generates a small, opposing electrophoretic velocity, causes the NAs to concentrate at the entrance to the contraction channel while all other components in the sample are flushed out from the device. Using the appropriate parameters, NAs can be retained in the device even though the mean expected flux due to the fluid flow greatly exceeds the opposing electrophoretic velocity. The mechanism for the trapping is known: upon entering the contraction channel, NA molecules stretch and orient themselves at an angle due to the imposed flow field and when the axial electric field is applied, the net orientation of the polymer drives a flux of molecules toward the bounding walls. The NAs become focused in a thin sheet next to the channel walls. Once at the walls, electrophoresis returns the NAs to the contraction channel entrance since the fluid flow decreases to zero at the walls (note that, in embodiments, electroosmosis may be suppressed by coatings or other methods, and in other aspects, alternate coatings can be use that can alter or otherwise enhance electroosmosis). Only NAs greater than approximately 5-10 k nucleotides/base pairs in length, although flow and electric field strengths can be tuned to selectively trap various lengths of NAs. Shorter NAs and other sample components like proteins, lipid, etc. are not trapped at the contraction entrance because they are smaller, rigid molecules with respect to higher molecular weight NAs.

After purified NAs are trapped at the contraction entrance they can then be fractionated by molecular weight in the same device by a protocol, such as the following embodiment: the electric field is turned off for a sufficient amount of time to allow NAs concentrated near the walls to diffuse back towards the center of the device. Once the extracted NAs are redistributed uniformly across the channel, the electric field is again applied again but at a lower strength then that used for the extraction. As before NAs are stretched and reorient themselves due to the imposed flow and electric fields. The velocity of the NAs in the vertical direction is dependent on the NA's size. Larger NA molecules will reach the wall faster than the smaller ones. Once at the walls, electrophoresis will retard the NAs velocity towards the outlet. Unlike in the extraction protocol, here the NAs are not returned to the contraction entrance because under the reduced electric field strength the fluid's velocity near the wall exceeds the electrophoretic velocity of the NAs (which is constant for NAs greater than a few hundred nucleotides). The length of NAs present can then be determined by measuring the time at which bands of NAs are eluted past a detector located near the exit of the channel. Shorter strands of NAs will elute before the longer strands.

The transport of nucleic acids through microfluidic devices relies primarily on electric fields, and less often on pressure-driven flows. Numerous studies have examined the detailed transport under a wide range of conditions for both types of fields; extensive reviews are available regarding the electrophoretic motion of nucleic acids and detailed studies of the conformation and transport of nucleic acids in shear flows are available. For capillaries much larger than the nucleic acids, the nucleic acids remain well-distributed across the transverse direction save for an excluded volume interaction with the walls and, in the case of pressure-driven flow with an electric field opposes to the pressure-driven flow, a small depletion layer caused by hydrodynamic effects.

To meet the needs of greater control and manipulation of nucleic acids within microfluidic channels, researchers have introduced higher-dimensional features to the devices. To weaken axial dispersion, channels have been curved to introduce a secondary, transverse flow that mixes the nucleic acids and introducing steps into a channel can separate nucleic acids by length. Although diffusion tends to distribute molecules uniformly across a capillary, hydrodynamic flows, for example when combined with an electric field, can produce strongly inhomogeneous distributions within a single cross-section. A flow field by itself produces a depletion layer towards the walls of the capillary, which hinders the detection of proteins and nucleic acid fragments by reducing the concentration at the walls of the capillary where the biomarkers can be located and thus current microfluidic biopolymer separation devices suffer from inefficient and often poor performance.

With that said, described herein are microfluidic devices that can concentrate and/or trap a particle, including without limitation biopolymers such as nucleic acids (RNA in particular), within a specific region of a microcapillary of a microfluidic device using a uniform electric field that can act in opposition to a fluid flow through the microcapillary. The microfluidic devices described herein can be used in series with other devices (for example devices that can lyse cells) and techniques that require a specific population and/or concentration of particles. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

In embodiments according to the present disclosure, it can be realized that electroosmosis can be controlled by altering the coating in microcapillaries of devices as described herein. In embodiments according to the present disclosure, electroosmosis can be enhanced with coatings (in particular coatings on one or more surfaces of the microcapillary channel). In embodiments according to the present disclosure, electroosmosis can be reduced with coatings (in particular coatings on one or more surfaces of the microcapillary channel).

In certain aspects of methods according to the present disclosure, the electric field can slowly be decreased in magnitude to control the isolation or elution of nucleic acids.

In certain aspects, microfluidic devices according to the present disclosure can have steps along the length with differing cross-sectional areas to isolate different nucleic acids in different areas of the device.

In certain aspects, detection moieties can be employed that allow for the easy distinction between nucleic acids, DNA and RNA, for example.

In certain aspects, detection moieties can be employed that allow for the detection of only RNA.

In other aspects, fluid tanks can be operably connected to a translation stage that allows for fluid pressure in the system to be altered based on height differences between the fluid inlet and outlet tanks.

### Microfluidic Particle Trap

Described herein are microfluidic devices capable of concentrating and/or trapping a particle in a region within the microfluidic device. The microfluidic devices described herein are configured to generate and/or comprise a fluid flow that is in opposition to an electric field, which can produce a hydrodynamic flow coupled with a recirculation flow within the microfluidic device that concentrates the particle in a region within the microfluidic device.

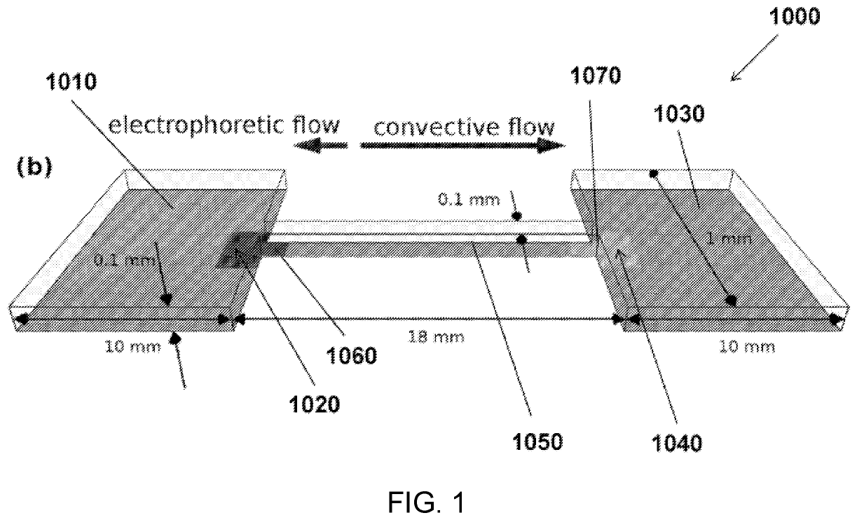

With the general concept described, attention is directed to FIG. 1, which shows one embodiment of a microfluidic trap 1000. The microfluidic trap 1000 can have an inlet area 1010, where the inlet area 1010 can have an entry region 1020. The entry region 1020 is the three dimensional area within the inlet area 1010 where the particle can concentrate. The microfluidic trap 1000 can have an outlet area 1030, where the outlet area 1030 can have an exit region 1040. The microfluidic trap 1000 can further have a microcapillary 1050, where one end of the microcapillary 1050 is fluidly coupled and/or physically coupled to the entry region 1020 of the inlet area 1010 and the other end of the microcapillary 1050 is fluidly coupled to and/or physically coupled to the exit region 1040 of the outlet area 1030. The point where the microcapillary 1050 meets the entry region 1020 can be referred to as the microcapillary inlet 1060. The point where the microcapillary 1050 meets the exit region 1040 can be referred to as the microcapillary outlet 1070.

The microcapillary 1050 and/or the inlet area 1010 and/or outlet area 1030 can be coated, either partially or completely, with a suitable neutral compound or polymer. Suitable neutral polymers include, but are not limited to, polyvinylpyrrolidone (PVP), polyvinyl alcohol (PVA), methyl cellulose, and non-cross-linked polyacrylamide. The coating can suppress electroosmosis and prevent electro osmotic flow. Electroosmotic flow can allow the particle to escape concentration and/or trapping by the microfluidic trap 1000 because the electroosmotic flow generated can be larger than the velocity of the electrophoretic flow.

In certain embodiments, the microcapillary 1050 and/or the inlet area 1010 and/or outlet area 1030 can be coated, either partially or completely, can be coated with a compound or polymer that can enhance electroosmosis and electroosmotic flow. Suitable coatings that can enhance electroosmosis and electroosmotic flow include, polycationic coatings, for example poly(N,N-dimethylacrylamide (PDMAC) or poly(diallyldimehtylammonium) chloride (PDADMAC).

The microcapillary 1050 and/or the inlet area 1010 and/or outlet area 1030 can further comprise one or more capture molecules, such as a nucleic acid that is complementary to a nucleic acid of interest, an antibody or aptamer, or other specific binding partner to a particle of interest. In particular, a capture molecule can be configured to bind specifically to a sequence of DNA or RNA (i.e. target sequence) desired to be isolated and/or detected by the user. Such target sequences of interest can be found, for example, in bioinformatic databases such as the National Institute of Health National Library of Medicine National Center for Biotechnology Information (NCBI).

In an embodiment, capture molecules as described herein are configured to specifically bind severe acute respiratory syndrome coronavirus 2 (SARS-CoV-2) viral RNA. Capture molecules that specifically bind SARS-CoV-2 can have a structure that is complementary to at least a portion of the SARS-CoV-2 genome (for example NCBI reference sequence NC_045512.2). In aspects, capture molecules can be configured to bind to specific portions of SARS-CoV-2 viral RNA, for example a portion encoding the surface or spike glycoprotein (for example NCBI Reference Sequence YP_009724390.1).

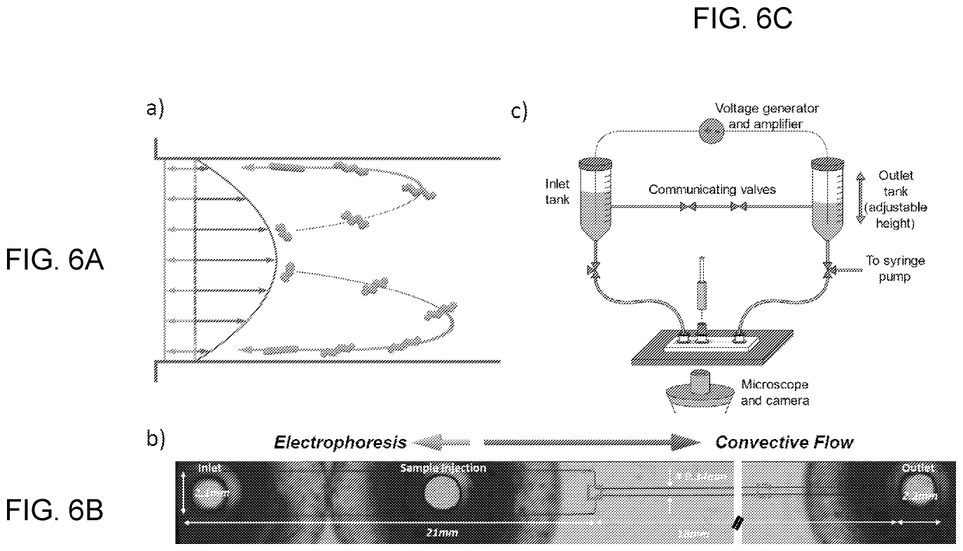

The microfluidic trap 1000 can be configured to generate an electrophoretic flow that is in opposition to a fluid flow through the microcapillary 1050. Devices and mechanisms to generate a fluid flow through a microfluidic device are generally known to those of skill in the art. Devices and mechanisms to generally apply a current through a microfluidic device are generally known to those of skill in the art. When an electrophoretic flow is generated in the microfluidic trap 1000 in opposition to a fluid flow in the microfluidic trap 1000, a particle, which is oriented at an angle to the fluid flow prior to the generation of an electrophoretic flow, the particle can be driven to the walls of the microcapillary 1050 and then recirculated back towards the entry region 1060. Without being bound by theory, it is believed that the net orientation of particles drives a flux of the particles perpendicular to the field lines (See e.g. FIG. 6A), which concentrates the particle at a stagnation point/region within the microcapillary 1050 and/or entry region 1020.

The width of the microcapillary can be greater than about 0.1 μm. In some embodiments, the width of the microcapillary 1050 can range from about 0.1 μm to about 2 mm. The microcapillary can be any suitable and/or desired length. In some embodiments, the length of the microcapillary can range from about 100 μm to about 1 m or greater. It will be appreciated that the length and width of the microcapillary 1050 can vary according to the size of particle desired to be concentrated and/or trapped. By altering the dimensions of the microcapillary 1050 it can be possible to tune the size and/or type of particle being concentrated and/or trapped. The microcapillary 1050 can be uniform in dimensions along its length. In other embodiments, the microcapillary 1050 can be tapered at one or both ends. In further embodiments, the microcapillary 1050 can comprise one or more steps where the width and/or height of the microcapillary 1050 changes (increases or decreases) abruptly to generate a step form in the shape of the microcapillary 1050. These changes in dimensions can further allow a user to tune the size and/or type of particle being concentrated/trapped. These changes in dimensions can also allow a user to tune the stagnation point where the particle is concentrated and/or trapped. It would be appreciated by the skilled artisan that the microcapillary 1050 can also be a variety of lengths with a variety of cross-sectional areas.

The skilled artisan would appreciate that the cross-sectional geometric shape of the microcapillary 1050 is not particularly limiting, and can be of a variety of geometric shapes. In an embodiment, the microcapillary 1050 is trapezoidal in geometric shape. In other embodiments, it may be circular in geometric shape. In other embodiments, it may be square or rectangular.

The inlet area 1010 can have a width that is at least greater than the width of the microcapillary 1050. In some embodiments, the inlet area 1010 can have a width ranging from about 1 mm to about 100 mm. The inlet area 1010 can have any suitable and/or desirable length greater than 0. In some embodiments, the length of the inlet area 1010 is greater than 0.1 μm. In other embodiments, the inlet area 1010 can have a length ranging from about 0.1 μm to about 1 m. The height of the inlet area 1010 can be any suitable and/or desired height greater than 0. In some embodiments, the inlet area 1010 can have a height ranging from about 1 μm to about 500 μm.

The outlet area 1030 can have a width that is at least greater than the width of the microcapillary 1050. In some embodiments, the outlet area 1030 can have a width ranging from about 1 mm to about 100 mm. The outlet area 1030 can have any suitable and/or desirable length greater than 0. In some embodiments, the length of the outlet area 1030 is greater than 0.1 μm. In other embodiments, the outlet area 1030 can have a length ranging from about 0.1 μm to about 1 m. The height of the outlet area 1030 can be any suitable and/or desired height greater than 0. In some embodiments, the outlet area 1030 can have a height ranging from about 1 μm to about 500 μm.

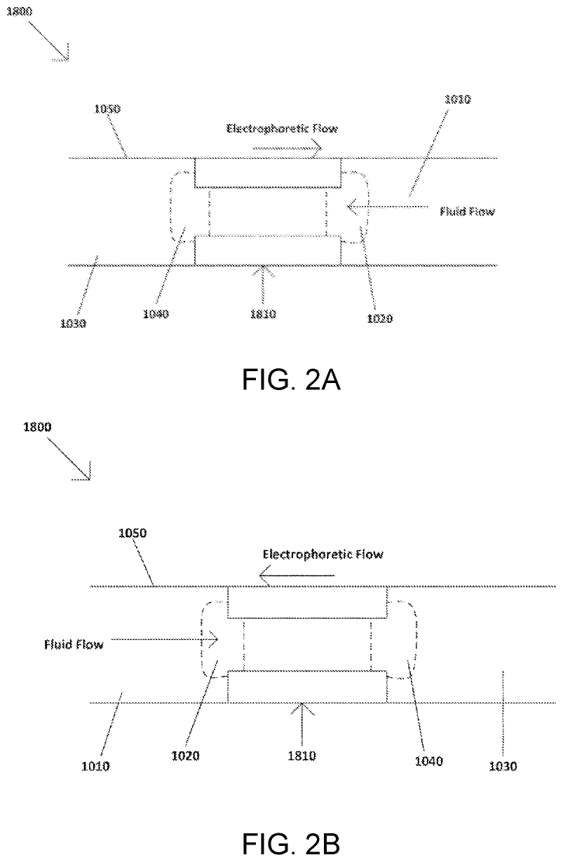

In some embodiments, a microfluidic trap can include a microcapillary that is constricted in one or more places along its length. In other words, in one or more places along its length, the cross-sectional area of the microcapillary is decreased. With this in mind, attention is directed to FIGS. 2A-2B, which show embodiments of a microfluidic trap 1800 where an inlet area 1010 and/or an outlet area 1030 are part of a microcapillary 1050. The constriction 1810 can generate an inlet area 1010 and/or outlet area 1030 within the microcapillary 1050. The length of the constriction 1810 can be greater than 0 but less than the entire length of the microcapillary 1050.

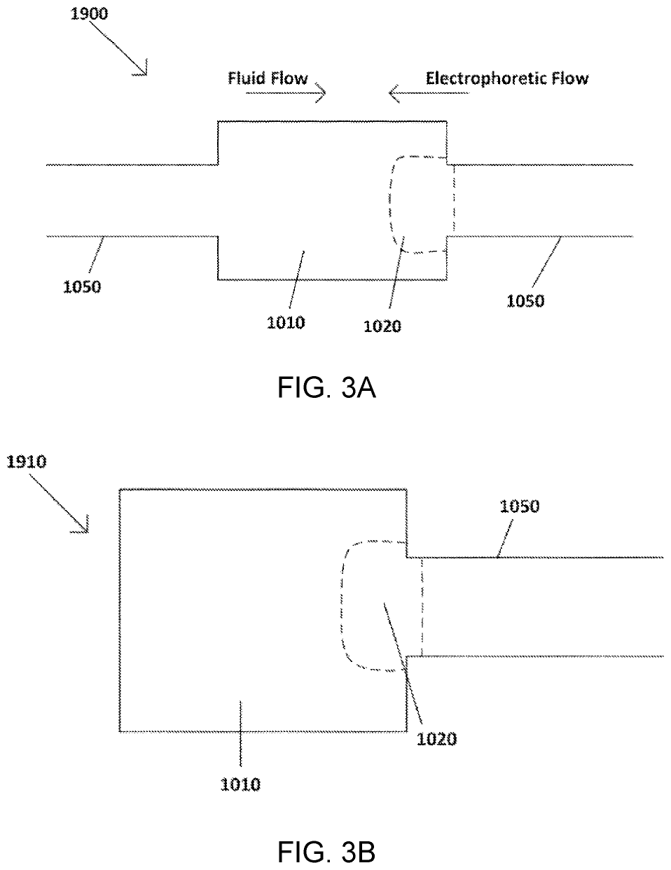

In other embodiments, the microfluidic trap does not comprise an outlet area. With that said FIGS. 3A-3B show embodiments of a microfluidic trap 1900, 1910 that does not comprise an outlet area. In these embodiments, the desired particles are concentrated at the entry region 1020 of the microfluidic trap 1900, 1910. The concentrated particles can be analyzed at that point or otherwise collected from the microfluidic trap 1900, 1910.

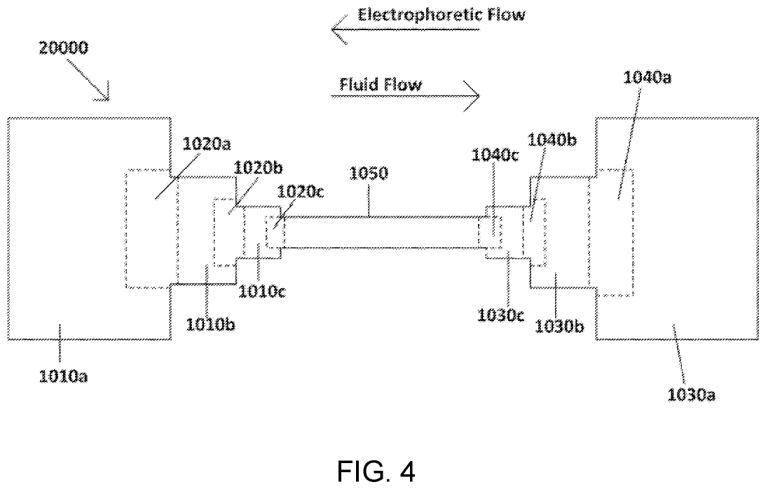

The microfluidic trap can comprise multiple inlet areas and/or outlet areas. With that said, FIG. 4 shows an embodiment of a microfluidic trap having multiple inlet areas and outlet areas. By generating step-wise or tapered changes in the width of the inlet area 1010, outlet area 1030 and/or microcapillary 1050, the particle can be concentrated at one or more of these entry regions 1020 a,b,c.

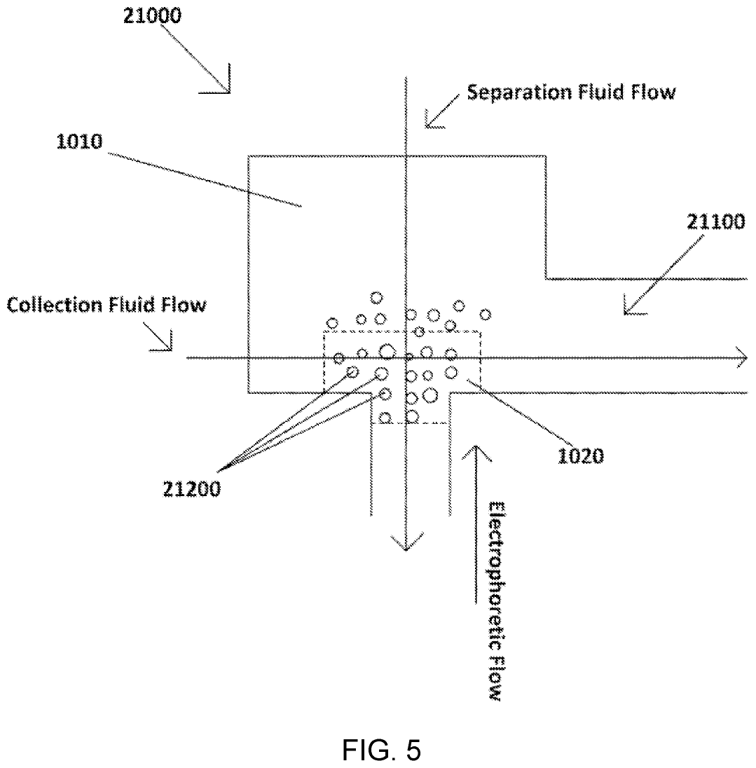

In some embodiments, it is desirable to collect the concentrated particles and directly transport the concentrated particles into an additional microfluidic device (such as in a multilayered microfluidic device) or other device for further particle processing or analysis. With that said, FIG. 5 shows an embodiment of a microfluidic trap 21000 configured to collect concentrated particles 21200 via a fluid flow (collection fluid flow) that is not parallel to the fluid flow used to drive sample particles into the microcapillary (separation fluid flow). The collection fluid flow can be applied at any angle that is not parallel to the separation fluid flow. The inlet area 1010 can be configured to comprise a collection port 21100 that can be fluidly and/or physically coupled to an additional microfluidic or other device. The collection fluid flow can drive the collected particles into the collection port 21100 and into the additional microfluidic or other device for further processing and/or analysis.

The microfluidic trap described herein can further comprise one or more detectors configured to detect one or more of the concentrated/trapped particles. Suitable detectors are generally known to those of ordinary skill in the art. The detector can be placed at any point in the microfluidic trap. In some embodiments, the detector can be placed at an entry region and/or collection point (also referred to herein as the stagnation point). In embodiments, detectors can be placed downstream in the microcapillary to sense the elution of trapped particles after releasing the trap.

### Systems

Described herein are systems for separating, isolating, and/or otherwise detecting nucleic acids. In embodiments, a system for isolating or detecting nucleic acids, comprises a microfluidic device as described herein and a buffer. In embodiments of systems as described herein, the buffer has an ionic concentration of about 0 mM to about 10 mM. In embodiments of systems as described herein, the buffer has a pH of about 6.5 to about 8.5. In embodiments of systems as described herein, systems further comprise an electric current generator configured to generate an electrophoretic flow through the microcapillary. In embodiments of systems according to the present disclosure, systems can comprise an inlet tank fluidically connected to the fluid inlet and an outlet tank fluidically connected to the fluid outlet. In embodiments of systems as described herein, system further comprise a valve in fluidic communication between the inlet tank and outlet tank. In embodiments of systems as described herein, systems can further comprise a syringe pump in fluidic communication with the fluid outlet. In embodiments of systems as described herein, systems can further comprise a valve in fluidic communication with the inlet tank and fluid inlet, upstream of the fluid inlet. In embodiments of systems as described herein, systems can further comprise a valve in fluidic communication with the outlet tank and fluid outlet, downstream of the fluid outlet. In embodiments of systems as described herein, systems can further comprise a pump to drive fluid motion through the system. In embodiments of systems as described herein, the microfluidic device, inlet tank, and outlet tank form a closed loop. In embodiments of systems as described herein, systems can further comprise a translational stage operably connected to the outlet tank. In embodiments of systems as described herein, systems can further comprise a computing controller. In embodiments of systems as described herein, systems can further comprise a micrograph image collecting apparatus. In embodiments of systems as described herein, the coating of the microcapillary suppresses osmotic flow. In embodiments of systems as described herein, the coating is a charge-neutral polymer.

### Methods of Using the Microfluidic Trap

Described herein are methods of using the microfluidic trap described herein. The microfluidic trap described herein can be used to concentrate and/or trap a particle at a particular point or region within the microfluidic trap. By applying an opposing fluid flow and electric fields to a solution comprising an amount of a particle in the microcapillary, the particle can be concentrated and/or trapped at a particular point or region within the microfluidic trap.

Particles contained in a pressure-driven fluid flow can orient themselves at an angle to the fluid flow. When an axial electric field is applied to the fluid, the net orientation of the particle can drive a flux of molecule perpendicular to the field lines. This is demonstrated in

FIG. 6A. Modest pressure drops (i.e. less than about 1 cm) and voltages (less than about 100 V) acting in opposition from one another, can drive a particle (e.g. DNA) to the walls of a microcapillary in a distance of less than 1 cm. The particle can become focused in a thin sheet (about 1 μm or less thick) next to the wall of the microcapillary. The thin layer of particle can contain almost, if not all of the particle present in the capillary. The thin layer of particle can be driven back against the fluid flow by the electrophoretic flow generated by the opposing electric field. By tuning the electric field to the fluid flow, a stagnation point can be generated at the microcapillary inlet (or other suitable region within the microfluidic trap). The stagnation point is where the particle can be concentrated. Because the particle is present and concentrated in a known area in the microfluidic trap, it is readily available for detection, extraction, or binding to a capture molecule located at or in the region of the stagnation point.

The method of concentrating and/or trapping a particle using a microfluidic trap as substantially described herein can comprise the steps of adding an amount of particle to the inlet area of a microfluidic device as substantially described herein (or a biological sample or sample derived from a biological sample, such as lysis products, comprising particles as described herein, for example nucleic acids), generating a fluid flow through the microcapillary of the microfluidic device and applying an electric field to the microfluidic device, where the electric field generates an electrophoretic flow that is in opposition to the fluid flow. The rate of fluid flow can range according to, inter alia, the dimensions of the microcapillary, the type of particle being concentrated and/or trapped, and the size of the particle being concentrated and/or trapped. In some embodiments, the rate of fluid flow can range from about 1 μm/s to about 500 mm/s. The fluid flow can be pulsed (i.e. repeated on/off cycles). The voltage of the electric field can vary according to, inter alia, the dimensions of the microcapillary, the type of particle being concentrated and/or trapped, and the size of the particle being concentrated and/or trapped. In some embodiments, the voltage can range from about 1 V/cm to about 1 kV/cm. The electric field can be a uniform electric field. In some embodiments, the electric field can be applied in pulses (i.e. repeated on/off cycles). The electric field can be applied for any suitable length of time. It will be appreciated that the length of time that the electric field is applied for can vary according to, inter alia, the dimensions of the microcapillary, the type of particle being concentrated and/or trapped, and the size of the particle being concentrated and/or trapped. Determining the appropriate length of time and/or voltage and/or fluid flow rate can be accomplished without undue experimentation by one of ordinary skill in the art.

The fluid flow and electric field can be generated simultaneously within the microfluidic trap. In other embodiments, the fluid flow and the electric field are generated serially from one another. For example, the fluid flow can be generated and allow the particle to enter the microcapillary prior to applying the electric field or the electric field can be applied prior to generating a fluid flow within the microfluidic trap.

The method can further comprise the step of concentrating and/or trapping the particle at the entry region of the inlet area of the microfluidic device. The method can further comprise the step of quantitating the amount of particle concentrated at the entry region of the inlet area. The method can further comprise the step of specifically binding one or more molecules of the particle to one or more capture molecules physically coupled to the microcapillary or inlet region of the microfluidic trap.

Methods as described herein can further comprise the step[s] of detecting one or more molecules of the collected particle. Methods of detecting particles are generally known in the art. In embodiments, detection methods as described herein comprise detecting a signal by laser or fluorescence microscopy, the signal a result of emission of a wavelength of light by a conjugate of molecules and a detection moiety. Examples of such conjugates include dyes and fluorophores that conjugate with or otherwise associate with nucleic acids, such as Acridine orange, ethidium bromide, Hoechst 33258, and oxazole yellow homodimer.

Such detection moieties can be included in the fluid phase of the buffer or sample, or by other means, for example a solid phase conjugated to part of the microfluidic device in the region of interest (or coating thereof).

The method can further comprise the step of collecting (i.e. removing one or more particle molecule from the point of collection) one or molecules of the particle after concentrating and/or trapping the particle within the microfluidic device. Collection of the particle can include without limitation, aspiration, adding/removing an electric field, and/or adding/removing a separation fluid flow (i.e. the fluid flow that drives the particles into the microcapillary for separation under an opposing electric field). In other embodiments, collection of the particle can include applying a collection fluid flow across the collection point (also referred to herein as the stagnation point), where the collection fluid flow is not parallel to the direction of the separation fluid flow. One embodiment that demonstrates collection of the particle using a collection fluid flow is shown in FIG. 4, which is discussed above. The concentrated particle can then be used in downstream methods and techniques generally known to those of ordinary skill in the art. These can include, without limitation, any form of PCR, nucleic acid sequencing, protein sequencing, and liquid chromatography.

In an aspect of the present disclosure, a method of product recovery can include: injecting a sample into the channel; applying specified flow rates and electric fields; running the protocol for a specified time (for example 20 or 40 minutes); at the end of the run time, cutting the electric field; imaging the DNA eluting from the channel; and converting the elution data into mass.

The devices described here can be tuned by altering the fluid flow, voltage, and dimensions/configuration of the devices described herein to separate specific particles within a suspension from undesired particles within the same suspension. For example, tuning can comprise altering the centerline velocity (v|0)down the longitudinal axis of the channel and the opposing electrophoretic velocity (vₑ and the ratio of (v|0/vₑ). The desired particles can be concentrated/trapped at the collection point when the fluid flow through the microcapillary is opposed by a uniform electric field. The undesired particles can pass through or not be collected or removed from the microfluidic trap.

In other aspects, tuning can comprise altering other parameters of operation, or components of the device and/or system. For example, tuning can comprise altering the electroosmotic properties of the microfluidic channel by application of a coating (which can either enhance or suppress electroosmosis). In other embodiments, tuning can comprise altering the ionic strength of the buffer that flows through or is otherwise present in the microfluidic channel. In other embodiments, tuning can comprise altering the pH of the buffer that flows through or is otherwise present in the microfluidic channel. In other embodiments, tuning may comprise altering the physical dimensions of shape of the microcapillary (for example length, width, cross-sectional area, geometry, and the like). The skilled artisan would understand that these parameters can be adjusted according to the nucleic acid[s] that are desired to be captured and/or detected with routine experimentation, and that physical characteristics of the desired capture molecules (size, charge density, GC ratio, chemical composition, nucleic acid type, etc) can be used to guide tuning of the parameters.

The input suspension for separation can be from any source prepared by any suitable method generally known in the art. In some embodiments, the suspension can comprise cell lysate that was processed outside of the microfluidic trap or within the microfluidic trap. In other embodiments, the suspension can be a PCR product in the PCR reaction solution. In these embodiments, the device can be used to separate out the desired PCR product from the primers, enzymes, and other components of the PCR reaction solution. In short, the devices and methods described herein can be used to separate any desired particle or group of particles from an undesirable one and, in some embodiments, prepare the solution or otherwise analyze the concentrated desired particle within the device itself.

In additional aspects of methods of using a microfluidic trap as described herein, it can be possible to isolate both DNA and RNA from a sample, and detect only either the DNA or the RNA present. This can be accomplished, for example, with enzymatic treatment. For example, after nucleic isolation, an RNase can be added to the buffer to selectively degrade any isolated RNA present in the sample (and only the RNA). In other embodiments, a DNase can be added to the buffer to selectively degrade any isolated DNA present in the sample (and only the DNA). This can also be accomplished by tuning the operating parameters and/or buffers of the devise and system. In other aspects, this can be accomplished with capture molecules conjugated to aspects of the devices and systems as described herein. Capture molecules can be employed to selectively bind DNA or RNA, while any non-bound nucleic acid can be washed out of the system with a buffer or other wash solution.

### EXAMPLES

Now having described the embodiments of the present disclosure, in general, the following examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Experimental evidence that DNA can be separated and concentrated from bovine serum albumin (BSA) in low-cost capillaries by the electro-hydrodynamic coupling between a pressure-driven flow and a parallel electric field is reported herein. Under opposing flow and electrophoretic velocities, a polyelectrolyte will migrate perpendicular to the field lines and towards the channel walls. In this work, we explore electro-hydrodynamic extraction of λ-DNA from BSA (30 mg/mL) using microliter samples (10 μL) containing small amounts of DNA (1 ng). Extraction efficiency was characterized by measuring DNA and BSA concentrations within the device using epifluorescence microscopy. Process operating conditions were optimized by varying the magnitude of the pressure gradient and electric field. Electro-hydrodynamic extraction (in a single-stage device) was found to recover approximately 0.5 ng of DNA (50%) while reducing the BSA concentration by four orders of magnitude with high throughput (20 minutes).

### Introduction

Analysis of genetic material is important to health care, food safety, forensic science, and other industries.¹ While many protocols exist for high purity extracts, most involve lengthy procedures, harsh reagents, and constant intervention.² In order to overcome these limitations, DNA extraction has become an area of significant interest to microfluidic researchers.³ Microfluidics is assumed to be the key to miniaturization and automation of genetic analysis within a micro total analysis system (μTAS).⁴

Existing microfluidic platforms for DNA extraction include those based on isotacophoresis, bifurcated field-flow fractionation, and ion selective membranes.⁵⁻⁸ However, these processes must deal with complications such as buffer gradients, intricate channel geometries, and embedded membranes, which lead to difficult device fabrication. A microfluidic DNA extraction system is described herein with the following advantages: 1) supplies sufficient quantities of inhibitor free DNA for downstream analysis; 2) inexpensive fabrication; 3) automated processing; and 4) potential to integrate with downstream processing to form a total analytical system. The extraction process is based on the transverse migration of DNA in combined shear and electric fields, a process named herein as “Electro-hydrodynamic Migration” (EHM). Highly purified DNA can be recovered within 15-20 minutes in a simple device fabricated from acrylic sheets using EHM-based extraction.

### Principle of Electro-Hydrodynamic Migration (EHM)

A flexible polyelectrolyte, such as DNA, is isotropically distributed on scales longer than the Kuhn length (100 nm), because this distribution maximizes the configurational entropy. Modest electric fields (up to a few hundred V/cm) do not significantly perturb the monomer distribution and the chain remains isotropic, or nearly so. In weak electric fields DNA migrates along field lines, with an electrophoretic velocity that is independent of its length beyond a few hundred base pairs. Small compact molecules, such as proteins, also migrate along electric field lines, so that more complex electrokinetic effects are usually needed to separate DNA from proteins. However, unlike proteins, which are relatively inflexible, DNA molecules can be easily extended by an applied shear flow into ellipsoidal (cigar-like) distributions of monomers. Even at relatively modest shear rates (less than 100 s⁻1) DNA can be stretched to lengths that are more than an order of magnitude larger than its equilibrium size. An important secondary effect of the shear flow is that the main axis of the elongated polymer is rotated so that it lies at an angle to the electric field direction.

An elongated charge distribution, for example a charged rod, has different electrophoretic mobilities perpendicular and parallel to its symmetry axis.⁹ If the axis lies at an angle to the electric field direction, this asymmetry in mobility leads to a net motion of the molecule perpendicular to the field lines as well as electrophoresis parallel to them. Transverse migration can be towards the center of the channel, when the electrophoretic and convective velocities are in the same direction, or towards the channel walls if the velocities are opposed.^{10,11} This effect is called electro-hydrodynamic migration (EHM) to emphasize its dependence on the coupling between the flow and electric fields. While there is an improving theoretical understanding of the underlying physics of EHM, a quantitative theory is still lacking.¹²⁻¹⁵ Nevertheless, transverse migration by EHM, leading to extensive accumulation of DNA at the walls of the channel, is an empirical fact, which has been confirmed by confocal microscopy.^{11,16,17} DNA that is both convected through a microcapillary and driven in the opposite direction by electrophoresis becomes highly localized in a thin (10 μm) layer next to the channel walls. The migration, indicated by the sketch in FIG. 6A, is very strong and the central region of the capillary is devoid of DNA. EHM sustains the large transverse concentration gradient against the molecular diffusion.

The distribution of DNA within a cross section of the microfluidic channel becomes highly non-uniform when subjected to simultaneous shear and electric forces.¹¹ Despite the large convective velocity (more than ten times the electrophoretic velocity), the net flux of DNA is against the flow and in the direction of the electrophoresis. This causes a recirculation of DNA molecules, illustrated by the arrows in FIG. 6A; a uniform distribution of DNA enters the channel by convection (blue arrows), migrates towards the walls by EHM (orange arrows), and then returns to the entrance by electrophoresis. In the majority of the channel cross section, the convective velocity exceeds the electrophoretic velocity, but in this region the DNA concentration is very small. Near the walls where the DNA concentration is very large, the electrophoretic velocity exceeds the fluid velocity and so the dominant DNA flux is towards the entrance.

When DNA molecules return past the entrance of the contraction capillary (solid red square in FIG. 6B) the fields (both shear and electric) drop by an order of magnitude, due to the sudden increase in cross section. By suitable combinations of flow and electric field the DNA can be accumulated at the channel entrance, as illustrated by the images in FIG. 6A. By focusing the epifluorescent image, it can be seen that the DNA has accumulated in a thin sheet near the upper and lower walls of the channel. This is in agreement with previous observations using confocal microscopy. 11 The DNA can be held for long periods of time by a treadmilling mechanism. Concentrated DNA near the walls diffuses towards the center of the entrance region and is then convected into the channel. Once in the channel it migrates to the walls, as illustrated in FIG. 6A and is then returned to the entrance by electrophoresis. Small amounts of DNA (of the order of 0.1-1 ng) can be trapped indefinitely by this means; larger amounts can be held for transient periods.

In contrast, proteins always follow the electric field. Since they are compact and relatively rigid molecules, they do not elongate in the flow so they will not undergo EHM. As a result, the distribution of proteins in the channel remains uniform and they are quickly eluted, leaving behind the purified DNA. The feasibility of this mechanism for DNA-protein separations is demonstrated in the results of the present disclosure. From the understanding of the physics of EHM, it seems likely that DNA and possibly RNA are the only biomolecules that will exhibit EHM; it is therefore a promising means for microfluidic purification of long nucleic acids.

Transverse migration can also be driven by the viscoelastic properties of the solution.¹⁸ Typically solutions used to create viscoelastic migration contain significant concentrations of added polymer (˜10% wt.) so that the normal stresses generate significant cross-stream forces. In our case, the buffer solution has no measurable viscoelastic response; it contains less than 1% wt. PVP (polyvinylpyrrolidone), which serves as a dynamic wall coating to reduce electroosmosis.¹⁹ Migration is still observed in the absence of added PVP, but recirculation is suppressed by the large electroosmotic flow towards the outlet. A significant practical difference is that viscoelastic migration is not selective because it is based on the properties of the fluid rather than the properties of individual molecules. In this paper it is demonstrated for the first time a microfluidic purification of DNA based on EHM—the protein concentration is reduced by four orders of magnitude in a single-stage device.

### EXPERIMENTAL SECTION

### Device Fabrication

The device was fabricated by fusing polymethylmethacrylate (PMMA or acrylic; Astra Products) sheets in a thermal press. First, two 200 micron thick and one ⅛″ thick, 1″×2″ sheets were cut using CO₂ laser ablation (Trotec-Speedy 360). An expansion/contraction channel (FIG. 6B) was cut into one of the thin sheets, and inlet, outlet, and sample injection ports were cut into the thick sheet. The three sheets were then cleaned with light detergent and 70% vol. isopropyl alcohol (Santa Cruz Biotechnology). The sheets were stacked between two glass plates and thermally welded in a dual heat plate manual press (Color King, China) at 230° F. for one hour, and were allowed to cool for 30 minutes before being removed from the press. Luer connectors (Nordson Plastics) were then attached to the inlet, outlet, and injection ports using epoxy. A rubber septum (Nordson Plastics) connected to the injection port enabled sample injection directly into the expansion section of the device, upstream of the channel.

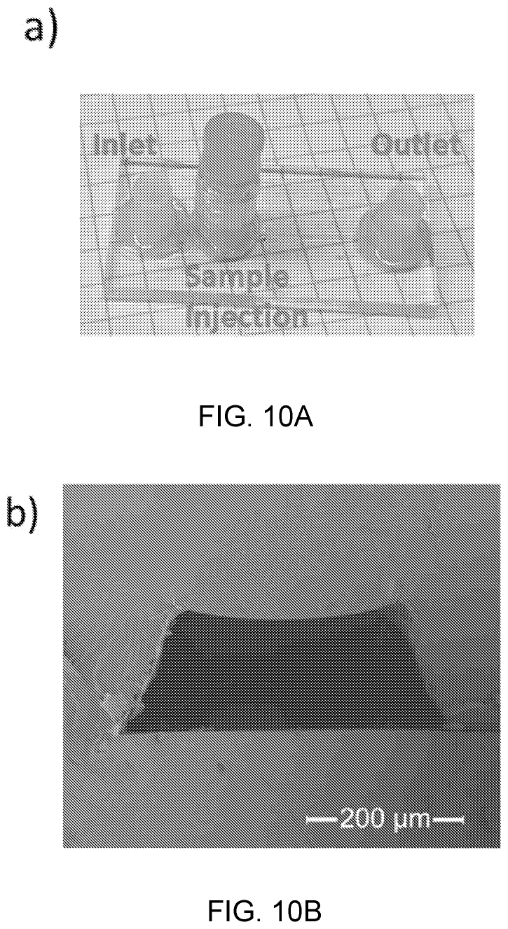

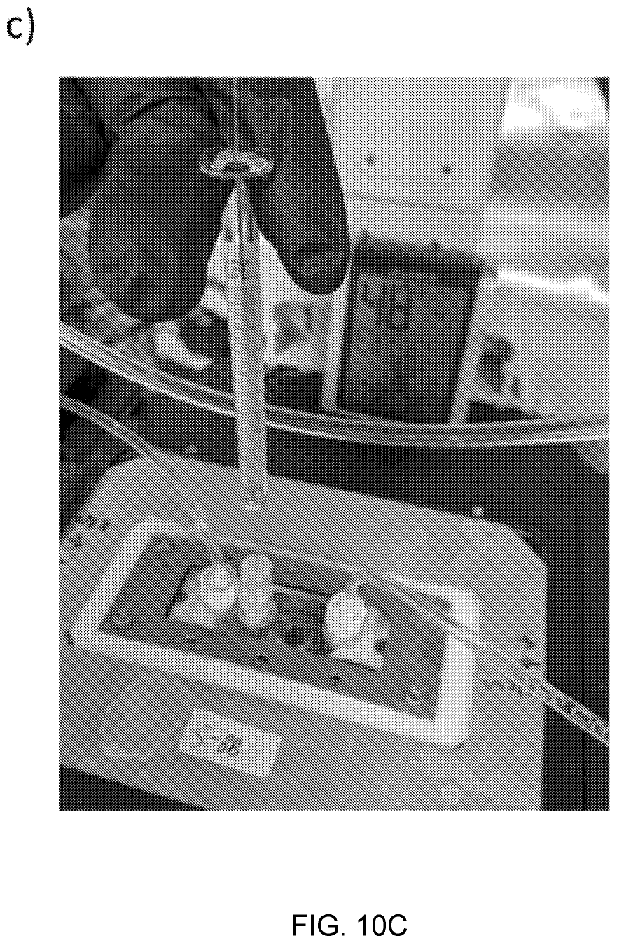

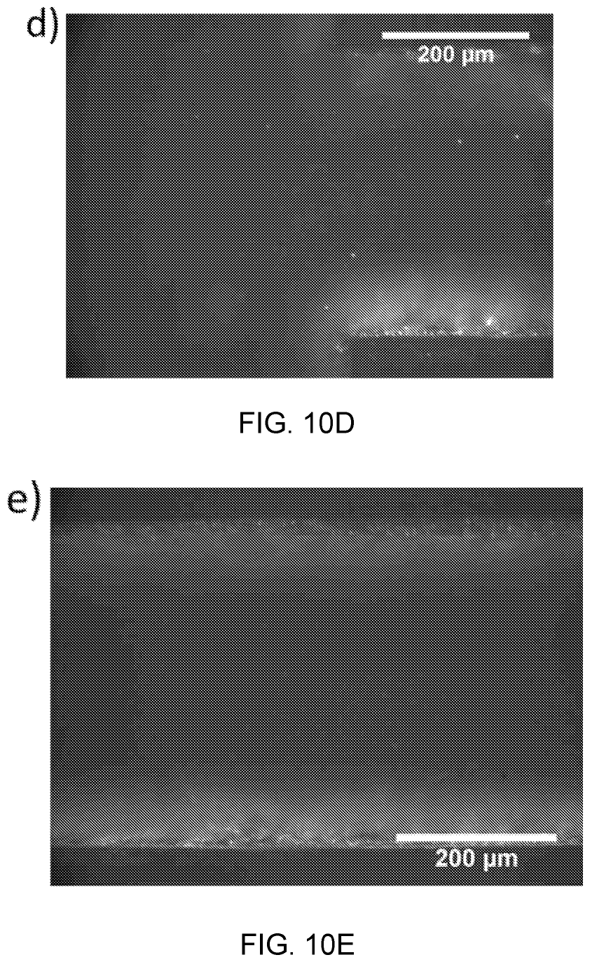

A brightfield micrograph of the assembled device is shown in FIG. 6B. The micrograph was constructed from 32 images taken at different locations with a Keyence BZ-X810 microscope (4× objective). The images were then stitched together using BZ-X Analyzer software. Additional brightfield micrographs, scanning electron micrograph, and digital images of the device are provided in the supporting information (FIGS. 10A-10E).

Channel features cut into PMMA sheets using CO₂ laser ablation yielded trapezoidal shaped channels (FIG. 10B), consistent with other reports.²⁰⁻²² Minimum and maximum widths of the contraction channel's cross-section were measured to be approximately 290 and 390 microns, respectively (average channel width of about 340 μm). The average depth of the device was about 150 μm. The height reduction from the 200 μm thickness of the original PMMA sheet is due to the light pressure applied during the thermal bonding process.

### Solution and Sample Preparation

Buffer solution was prepared by diluting concentrated (100×) Tris-EDTA (TE) buffer (Sigma) with deionized water (Barnstead Nanopure, 18.2 MΩ·cm) to a standard 0.25× solution containing 2.5 mM Tris-HCl and 0.25 mM EDTA. Neutral polymer, polyvinylpyrrolidone (PVP), with a molecular weight of 40 kDa (Sigma), was added to a concentration of 0.5% wt. to reduce electroosmosis towards the outlet. The buffer solution (pH about 7.3) was then filtered (0.22 μm, PES) and degassed under vacuum for 30 minutes prior to use.

Diluted TE buffer was chosen as process buffer because it is commonly used for preservation of nucleic acids. The concentration of TE was reduced from those typically used for DNA storage to ensure a low ionic strength (8 mM for 0.25×TE) of the flowing buffer solution.¹⁹ A neutral polymer (PVP) was added to the process buffer to act as a dynamic channel coating, in order to reduce electroosmosis within the channel. Acrylic capillaries have an inherent electroosmotic flow (EOF) which is comparable to those of silica and glass capillaries.²³ PVP was selected to reduce the EOF as its use has been well established for the elimination of EOF in glass and silica capillaries.^{24,26} Its use as a sieving matrix in capillary electrophoresis for DNA separations suggests it has a minimal effect on the DNA.²⁶

DNA/BSA mixtures were prepared by adding λ-DNA (New England Biolabs, 48 kbp) and BSA (Sigma, 66.5 kDa) at concentrations of 0.1 ng/μL and 30 mg/mL, respectively, to a 25×TE buffer solution. The concentration of protein is approximately equal to typical physiological concentrations while the DNA concentration is roughly two orders of magnitude lower than those commonly encountered in blood samples.^{27,28} BSA was chosen as a model protein to separate from DNA because albumin comprises the majority of serum proteins and holds a negative charge, the same as DNA, under the neutral pH conditions used here and found in blood samples.²⁷

The DNA/BSA mixtures were fluorescently tagged to quantify the concentration of DNA and BSA. YOYO-3 (1 mM stock solution in dimethyl sulfoxide, Invitrogen) DNA intercalating dye was used to label the DNA at a ratio of four base pairs to one dye molecule. BSA was marked by mixing unlabeled BSA with FITC-BSA (Sigma) at a concentration of 2.5% wt. YOYO-3 emits red light (612/631 nm excitation/emission) while FITC emits green light (494/520 nm). FITC-BSA was only used in experiments to measure BSA concentration, to eliminate the interference with DNA quantification that is associated with crosstalk from high levels of FITC-BSA fluorescence.

In some cases, salt was added to the DNA/BSA samples to study the effect of ionic strength on the separation process. Salt containing mixtures were prepared by adding sodium chloride (NaCl, Sigma) to give a final NaCl concentration of 150 mM.

### Sample Injection and Extraction Protocol

The acrylic channel was mounted into the experimental setup as indicated by the schematic in FIG. 6C and the reservoirs were filled with approximately 40 mL of the process buffer. Fluid flow within the channel was driven by a height difference between the two reservoirs. The flow rate was adjusted by lowering the height of the outlet reservoir using a translation stage (Thorlabs). The stage was adjusted by a stepper motor controlled by an open source microcontroller (Arduino Leonardo board).

Electric fields in the device were generated by applying up to 250 volts across stainless steel electrodes placed in the reservoirs (Agilent 3321A voltage generator and Trek 2220 amplifier). The maximum field within the channel was about 140 V/cm, with nearly all the potential drop occurring within the 1.8 cm long contraction channel.¹¹

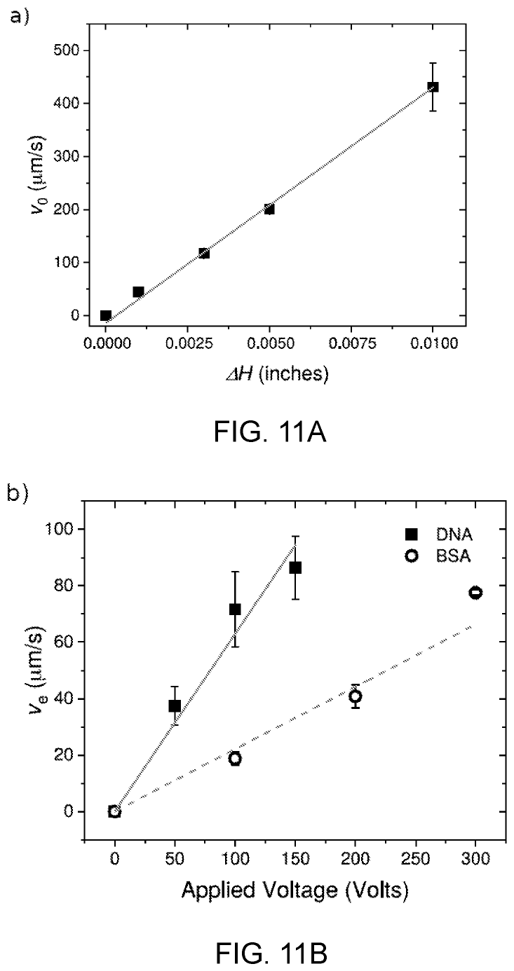

The centerline velocity of the fluid in the channel, v0, was measured by tracking the displacement of fluorescent latex beads (PolyScience) as a function of height difference between the reservoirs. A linear correlation between centerline particle velocity and height difference was observed (FIG. 11A) with a slope of 44 mm/s per inch height difference, which was used to control the fluid velocity along the centerline of the channel; typical flow rates were on the order of 1 mL/hr.

The electrophoretic velocity of DNA in the channel, vₑ, was found in a similar manner to v₀. The electrophoretic velocity of DNA was found to be 0.6 μm/s per volt for the buffer solution used in the experiments. A different procedure was used to measure the electrophoretic velocity of BSA due to the small size of BSA molecules. Instead of tracking individual BSA particles, the displacement of a slug of FITC-BSA in the channel under an electric field was measured as a function of time. The electrophoretic velocity of the BSA solution was found to be 0.2 μm/s per volt. Linear correlations between electrophoretic velocity and applied voltage for DNA and BSA are shown in FIG. 11B.

Extraction experiments were performed as follows. First, fluid levels were equilibrated by opening communicating valves located on the line connecting the two reservoirs (see FIG. 6C) to ensure no-flow within the channel prior to beginning the experiments. Once equilibrium was achieved, the communicating valves were closed. Next, 10 μL of sample mixtures were injected directly into the expansion section of the device with a chromatography syringe (Hamilton 80000 1701N Syringe) and septum as illustrated in the digital image in FIG. 10B. After sample injection, the syringe was removed, and an electric field and pressure driven flow were imposed. Fluorescent images acquired during the extraction process were used to quantify DNA and BSA concentrations within the channel.

### Imaging and Measurement Protocol

Epifluorescence images were captured with a Nikon Diaphot 200 inverted microscope equipped with a QImaging Retiga SRV CCD camera. All images were collected using a 200 ms exposure time and a gain of 39 unless stated otherwise. A Nikon ELWD 20x/0.4 objective was used for imaging. In epifluorescence mode, Nikon B-1A (blue) and Nikon G-1B (green) excitation filters were used to image FITC-BSA and YOYO-3, respectively. A mercury lamp (Nikon HB 10101AF) with an Osram HBO Mercury short-arc lamp 103W/2 bulb was used to stimulate the fluorophores. Illumination of the device was controlled by a motorized shutter actuated by an open source microcontroller (Arduino Uno board). A custom MATLAB (MathWorks, 2014) based controller program was used to control all operations associated with the experiment, including shutter actuation, image acquisition, reservoir height difference, and applied voltage.

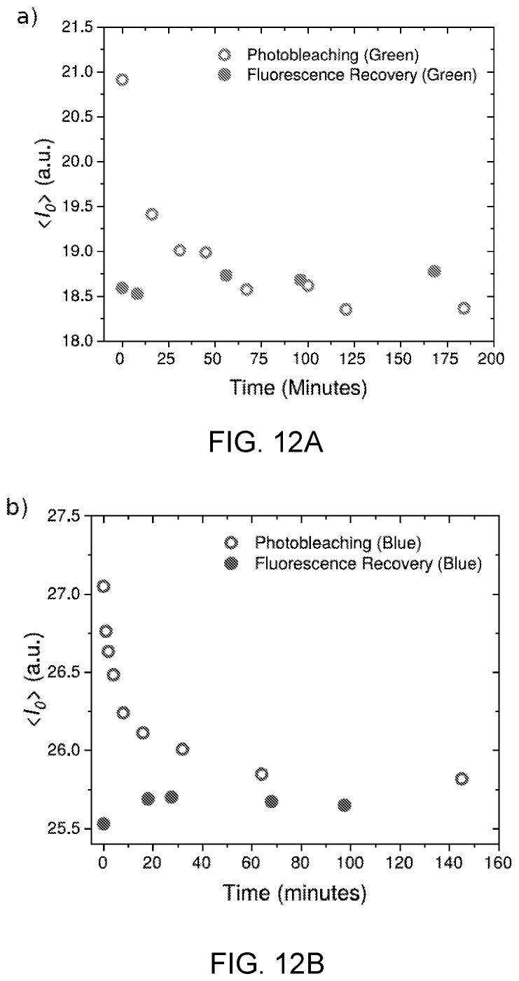

Prior to an experiment or calibration, the microscope objective was focused either at the entrance to the contraction channel or downstream near the exit region (the solid and dashed red squares in FIG. 6B, respectively) and illuminated by the mercury lamp for 2 hours to suppress the autofluorescence signal emitted by the acrylic. As the skilled artisan would understand, the illumination step (or presence thereof) will depend on the material used for device construction. Furthermore, this step can be altered by changing light wavelength, source, or timing depending on the material used for the device (for example the specific type of acrylic) as it would be understood that any inherent autofluorescence signal will be dependent upon the specific material used for device construction. Baseline average pixel intensity ( [symbol] I₀ [symbol] ) as a function of irradiation and recovery times are provided in FIGS. 12A-12B. Acrylic devices have high background signals due to PMMA's intrinsic autofluorescence29 It is found that irradiating the channel with the mercury lamp in advance of measurements suppresses the autofluorescence signal. While the intensity of PMMA's background signal does recover after photobleaching, it is at rates slow enough that the background signal recovery can be neglected in the calculations used here.

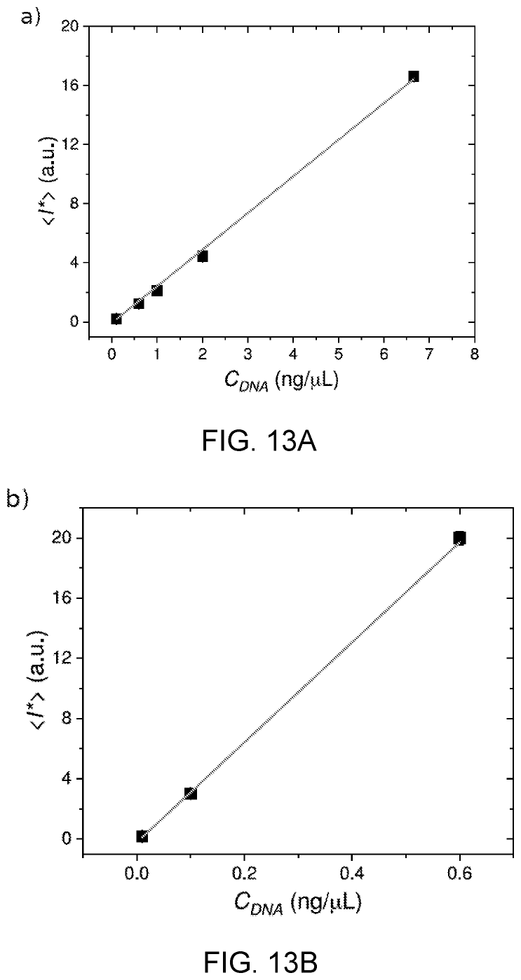

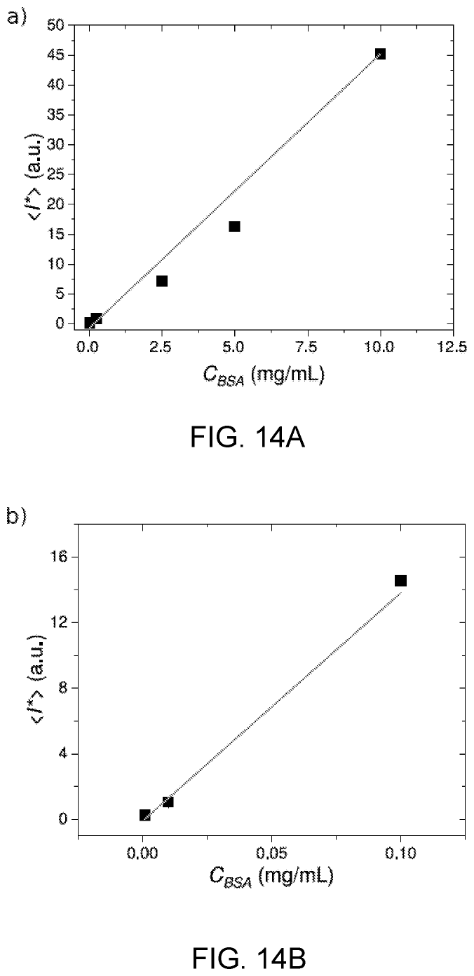

The fluorescent intensity relationship to concentration was calibrated by injecting approximately 20 μL of solution with known concentration directly into the device. The calibration solutions were convected past the field of view using a syringe pump (Harvard Apparatus) at a rate of approximately 0.9 mL/hr. The intensity of each solution was determined as an average over the same viewing window used in the experiment. The average pixel intensities ( [symbol] I [symbol] ) were then corrected by subtracting off the average baseline intensity ( [symbol] I₀ [symbol] ) of pure buffer solution flowing past the field of view. The baseline corrected average intensity, [symbol] I* [symbol] = [symbol] I [symbol] − [symbol] I₀ [symbol] , was converted to concentration using the calibration lines C=β [symbol] I* [symbol] . The calibration slopes, β, relating concentration of DNA (ng/μL) and FITC-BSA (mg/mL) to average fluorescence pixel intensity were taken daily to account for degradation in the mercury lamp's output. Sample calibration curves for DNA and BSA are provided in supporting information FIGS. 13A-13B and FIGS. 14A-14B, respectively.

DNA trapping kinetics were measured at the entrance to the contraction channel (solid red square in FIG. 6B). Images illuminated by the green excitation filter were taken at four evenly spaced intervals to minimize photobleaching of the YOYO-3 fluorophore. An 8× neutral density filter and camera gain setting of 30 were used to ensure accurate measurement of high concentration DNA without saturating the individual pixels. In these measurements, only DNA was fluorescently tagged, to avoid interference signal emitted by FITC-BSA when using the green excitation filter.

BSA elution rates were measured in a similar fashion to DNA trapping kinetics. Two different sets of images were acquired with different neutral density filters to capture the full range of BSA concentrations, in both cases using the blue excitation filter. High concentration BSA eluting past the contraction channel entrance (solid red square in FIG. 6B) was imaged by illuminating the device every 30 seconds for 20 minutes using the 8× neutral density filter. In subsequent experiments, trace concentrations of BSA were imaged four times every five minutes without any neutral density filtering. Infrequent illumination and imaging mitigates photobleaching which can otherwise lead to inaccurate measurements at lower concentrations. The total amount of DNA trapped during the separation process was measured by an elution analysis described previously. 30 The DNA extraction was performed on an injected DNA/BSA mixture by imposing a pressure driven flow and electric field. At a specified time, the electric field was turned off, releasing the accumulated DNA from the device. The eluting DNA was then imaged as it exited the contraction channel (dashed red square in FIG. 6B). The shutter was left open for the full duration of the experiment to photobleach any DNA that may have adsorbed on the channel walls prior to the imaging sequence beginning. The accumulated DNA upstream of the viewing window is only illuminated as it exits the channel. The total mass of DNA trapped during the extraction was estimated from the mass flow rate

Mₒᵤₜ≈Q∫ₜ^{τ}C(t)dt (Eq. 1)

where τ is the time required to elute all of the DNA from the device. Here τ=120 s is found to be sufficient. The volumetric flow rate is

$$Q = \frac{{v}_{0} {A}_{X}}{f} ,$$

where the centerline fluid velocity is v₀, the cross-sectional area of the channel Aₓ about 0.05 mm², and f is a shape factor (f about 1.5 for a trapezoidal channel). The concentration of DNA passing the field of view as a function of time is C(t). Equation (1) assumes a uniform distribution of DNA across the channel as it passes the detector; there is no electric field during these measurements.

Between each experiment, the channel was rinsed for five minutes by redirecting the flow via a control valve to the syringe pump, which drew fresh buffer solution from the inlet tank through the channel at a flow rate of 6.9 mL/hr. The shutter was left open during the rinse cycles to photobleach any fluorophore adsorbed to the walls and to repress the acrylic's autofluorescence signal.

### Results and Discussion

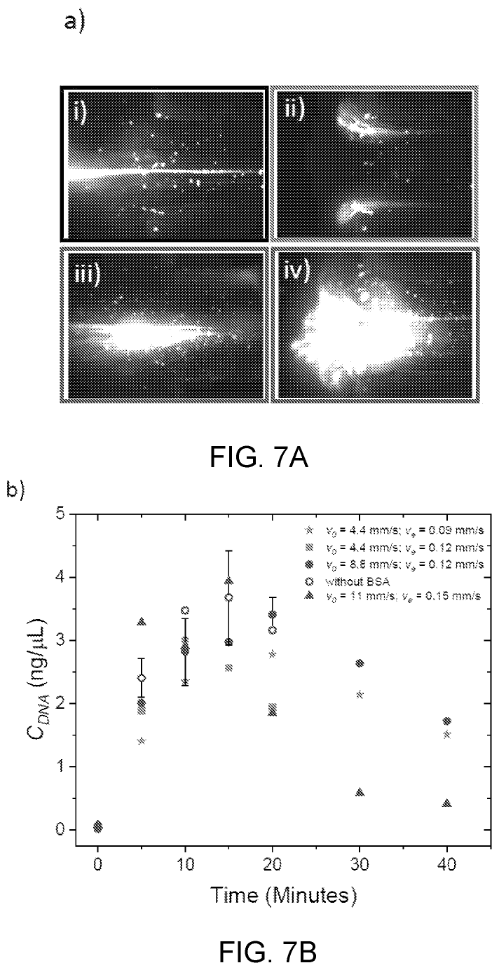

DNA trapping at the contraction channel entrance of an embodiment of a device as described herein was first tested. DNA concentration as a function of processing time for four combinations of fluid centerline velocity, v₀, and opposing DNA electrophoretic velocity, vₑ, are shown in FIG. 7B. Example fluorescent micrographs of DNA concentration patterns for each parameter combination are shown in FIG. 7A. As a control, trapping kinetics of DNA samples were measured in the absence of BSA (open blue circles, FIG. 7B) to verify that EHM and subsequent trapping of DNA occurs in the acrylic device with different dimensions with respect to previous work carried out in fused silica channels (without BSA).^{11,30} As expected, the DNA focused at the contraction channel entrance in thin sheets near the upper and lower walls, where the shear rate is largest. DNA trapping was optimized at ratios of flow and field strengths similar to previous work using silica channels.³⁰

It should be noted that EHM and consequent trapping of DNA is still observed when PVP is absent from the process buffer, but enrichment is reduced by an order of magnitude for the convective and electrophoretic velocities used here. This result indicates that dynamic coating with dilute PVP is effective at reducing electroosmosis in acrylic channels. The observed decrease in trapping efficiency stems from the electroosmotic contribution to the fluid velocity near the wall, which can exceed the opposing electrophoretic velocity of DNA. EHM is independent of electroosmosis but the electrophoretic recovery is not. Further work would be required to develop EOF suppressing static coatings to facilitate extractions without neutral polymer additives in the process buffer.

Comparing accumulation kinetics between samples containing mixtures of DNA and BSA to those containing pure DNA suggests that the presence of BSA has little effect on the EHM accumulation process. The data shows DNA concentrations reaching a maximum in 15 minutes for pure DNA and 20 minutes for DNA/BSA mixtures, with approximately equal maxima. One possible explanation in the delayed accumulation could be the higher viscosity of BSA containing samples reduces the EHM velocity in the directions perpendicular to the flow decreasing the rate at which DNA is able to reach the walls. The DNA concentration measured at the channel entrance for the mixture samples reaches a maximum in 15-20 minutes before dropping approximately 50% for most conditions examined. Previous experience suggests that microfluidic devices of comparable size can become saturated with DNA at amounts beyond a few tenths of a nanogram.³⁰ It is suspected that not all of the injected DNA can be trapped indefinitely although this depends on device dimensions and operating conditions. At the highest electric field (purple triangles, FIG. 7B) the DNA is being drawn back into the channel, upstream of the observation window. Here the amount of DNA in the device may be significantly more than what is observed near the channel entrance. A peak concentration of approximately 4 ng/μL DNA is measured at the entrance for v₀ of 11 and 8.8 mm/s and vₑ of 0.15 and 0.12 mm/s, respectively for DNA samples containing unlabeled BSA. This is a 40-fold increase of the initial concentration, 0.1 ng/μL injected into the device.

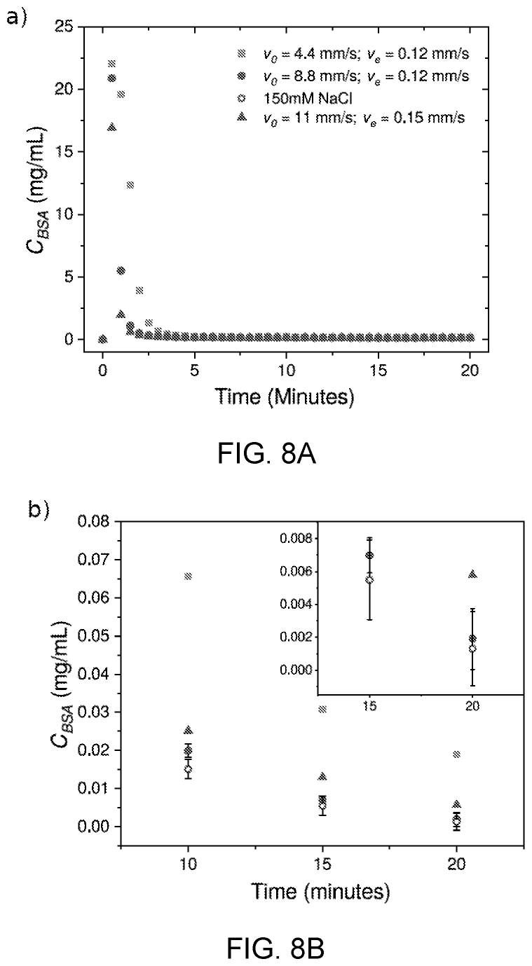

The elution rates of FITC-labeled BSA were measured at the channel contraction entrance as shown in FIGS. 8A-8B. FIG. 8A (8× neutral density filter) shows high BSA concentrations eluting, while trace BSA concentrations shown in FIG. 8B were acquired without neutral density filtering. DNA was fluorescently tagged in all BSA probing experiments to verify that DNA trapping had occurred after BSA measurements were taken. DNA fluorescence did not interfere with the FITC-BSA signal.

BSA is rapidly eluted from the channel and reduced to trace concentrations in under three minutes. At least some portion of the remaining BSA is likely adsorbed to the walls of the PMMA channel. The slightly reduced BSA concentration measured when samples salted with 150 mM NaCl are injected into the channel are consistent with other reports of reduced non-selective protein adsorption to capillary walls at higher solution ionic strengths.³¹ A reduction of BSA impurities from 30 mg/mL to 3±1 μg/mL (in a single-stage device) has been demonstrated using the optimal combination of vₒ=8.8 mm/s and vₑ=0.12 mm/s.

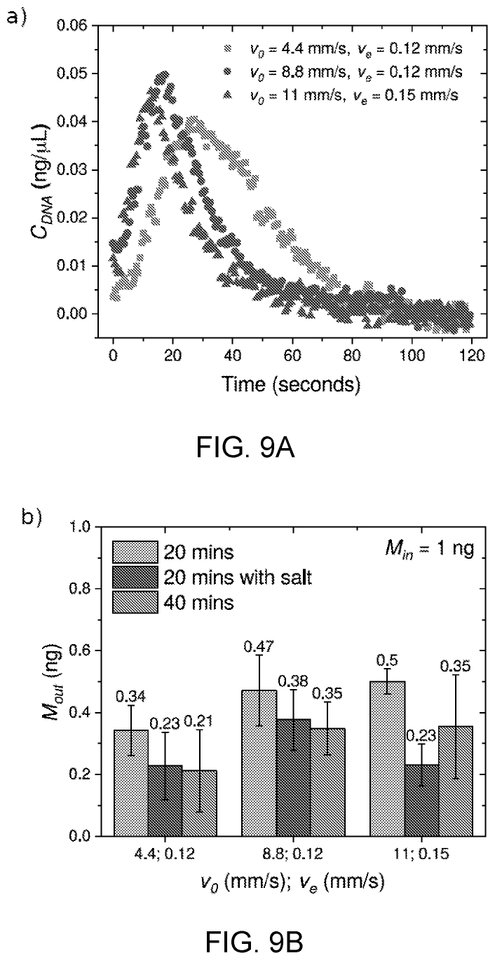

The total amount of DNA retained in the device was estimated using the protocol described above for the three highest field strengths, with both salt free and salted DNA/BSA mixtures after 20-40 minutes of extraction. Typical DNA concentration profiles for trapped DNA flushed from the device are shown in FIG. 9A. A concentration pulse passes through the viewing window soon after turning off the electric field. Subsequently the concentration decays to zero, indicating that all the DNA was flushed from the device. The total DNA that was trapped in the device was calculated using equation (1). Results are shown in FIG. 9B. Separation with salted samples containing dissolved NaCI at a concentration of 150 mM were measured to better simulate physiological conditions characteristic of blood serum samples. Extraction times of 20 and 40 minutes were used to determine if the decreases in DNA concentration at the contraction entrance observed at long times are in fact due to DNA escaping from the device. Sample mixtures of volume 10 μL containing 1 ng of DNA were used in all the DNA yield measurements. As can be seen in FIG. 9B, a maximum of 0.5 ng DNA was retained, corresponding to a 50% yield.

Yield measurements for salted samples (blue bars, FIGS. 9A-9B) are slightly reduced compared to salt free samples as expected due to electrostatic screening of the DNA's charged phosphate backbone. Salt concentrations in the bulk solution above 50 mM were previously shown to suppress EHM almost entirely.¹⁹ Here the salt concentration in the sample was initially high, but was rapidly diluted so that EHM can occur. DNA yields after a 40-minute extraction (red bars, FIG. 9B) were found to vary significantly from run to run and were on average lower than those from a processing time of 20 minutes. The large fluctuations in yield at longer times suggests that DNA slowly leaks from the device as it cycles through the EHM treadmilling mechanism described in FIG. 6A; this may be sensitive to the sample injection which varies considerably from run to run.

All DNA trapped and flushed from the device is of high purity, containing only trace concentrations of BSA similar to those shown in FIG. 8B. This is confirmed by observing no measurable pulses in fluorescent intensity when identical measurements were performed using mixture samples containing FITC-BSA.

### Conclusions

For the first time, EHM-based extraction, enrichment, and detection of nucleic acids from bovine serum albumin has been demonstrated. This technique relies on a pressure driven flow applied simultaneously with an electric field to create inhomogeneous concentration distributions of DNA within a microcapillary. EHM-based extraction relies on cross-stream migrations of DNA towards bounding walls; it uses no external mass separating agents, except for a dilute dynamic coating of neutral polymer for electroosmosis reduction. Dynamic coatings could potentially be avoided altogether by employing EOF-suppressing static coatings for acrylic surfaces.

Electro-hydrodynamic extraction has been shown to reduce protein concentrations in DNA/BSA mixtures 10,000 fold in a single-stage device. The process can be applied to microvolume samples (10μL) in simple, low-cost, acrylic channels; the channels are extremely durable as evidenced by a single device being used for over 100 injection experiments.

Purified DNA can be obtained quickly (20 minutes) in significant yields (50%). EHM-based purification has the potential for integration with other microfluidic operations for complete genomic analysis.

### REFERENCES

(1) Guttmacher, A. E.; Porteous, M. E.; McInerney, J. D. Educating health-care professionals about genetics and genomics. Nat. Rev. Genet. 2007, 8, 151_157.

(2) Chacon-Cortes, D.; Gri_ths, L. R. Methods for extracting genomic DNA from whole blood samples: current perspectives. J. biorepos. sci. appl. med. 2014, 2, 1_9.

(3) Kim, J.; Johnson, M.; Hill, P.; Gale, B. K. Micro_uidic sample preparation: cell lysis and nucleic acid puri_cation. J. Integr. Biol. 2009, 1.

(4) Aroa, A.; Simone, G.; Salieb-Beugelaar, G. B.; Kim, J. T.; Manz, A. Latest Developments in Micro Total Analysis Systems. Anal. Chem. 2010, 82, 4830_4847.

(5) Persat, A.; Marshall, L. A.; Santiago, J. G. Puri_cation of Nucleic Acids from Whole Blood Using Isotachophoresis. Anal. Chem. 2009, 81, 9507_9511.

(6) Qu, Y.; Marshall, L. A.; Santiago, J. G. Simultaneous Puri_cation and Fractionation of Nucleic Acids and Proteins from Complex Samples Using Bidirectional Isotachophore-sis. Anal. Chem. 2014, 86, 7264_7268.

(7) Zhang, C.; Sun, G.; Senapati, S.; Chang, H.-C. A bifurcated continuous_eld-_ow fractionation (BCFFF) chip for high-yield and high-throughput nucleic acid extraction and puri_cation. Lab Chip 2019, 19, 3853_3861.

(8) Ouyang, W.; Li, Z.; Han, J. Pressure-Modulated Selective Electrokinetic Trapping for Direct Enrichment, Puri_cation, and Detection of Nucleic Acids in Human Serum. Anal. Chem. 2018, 90, 11366_11375.

(9) Chen, S. B.; Koch, D. L. Electrophoresis and Sedimentation of Charged Fibers. J. Colloid Interf. Sci. 1996, 180, 466_477.

(10) Arca, M.; Butler, J. E.; Ladd, A. J. C. Transverse migration of polyelectrolytes in micro_uidic channels induced by combined shear and electric_elds. Soft Matter 2015, 11, 4375_4382.

(11) Arca, M.; Ladd, A. J.; Butler, J. E. Electro-hydrodynamic concentration of genomic length DNA. Soft Matter 2016, 12, 6975_6984.

(12) Usta, O. B.; Butler, J. E.; Ladd, A. J. C. Transverse Migration of a Con_ned Polymer Driven by an External Force. Phys. Rev. Letters 2007, 98.

(13) Butler, J. E.; Usta, O. B.; Kekre, R.; Ladd, A. J. C. Kinetic theory of a con_ned polymer driven by an external force and pressure-driven_ow. Phys. Fluids 2007, 19.

(14) Kekre, R.; Butler, J. E.; Ladd, A. J. C. Role of hydrodynamic interactions in the migration of polyelectryolytes driven by a pressure gradient and an electric_eld. Phys. Rev. E 2010, 82.

(15) Ladd, A. J. C. Electrophoresis of sheared polyelectrolytes. Mol. Phys. 2018, 116, 3121_3133.

(16) Zheng, J.; Yeung, E. S. Anomalous Radial Migration of Single DNA Molecules in Capillary Electrophoresis. Anal. Chem. 2002, 74, 4536_4547.

(17) Zheng, J.; Yeung, E. S. Mechanism for the Separation of Large Molecules Based on Radial Migration in Capillary Electrophoresis. Anal. Chem. 2003, 75, 3675_3680.

(18) Ranchon, H.; Malbec, R.; Picot, V.; Boutonnet, A.; Terrapanich, P.; Joseph, P.; Le-ichle, T.; Bancaud, A. DNA separation and enrichment using electro-hydrodynamic bidirectional_ows in viscoelastic liquids. Lab Chip 2016, 16, 1243_1253.

(19) Montes, R. J.; Ladd, A. J. C.; Butler, J. E. Transverse migration and micro_uidic concentration of DNA using Newtonian bu_ers. Biomicro_udics 2019, 13.

(20) Islam, M. M.; Loewen, A.; Allen, P. B. Simple, low-cost fabrication of acrylic based droplet micro_uidics and its use to generate DNA-coated particles. Sci. Rep. 2018, 8.

(21) Nayak, N. C.; Lam, Y. C.; Yue, C. Y.; Sinha, A. T. CO2-laser micromachining of PMMA: the e_ect of polymer molecular weight. J. Micromech. Microeng 2008, 18.

(22) Cheng, J.-Y.; Wei, C.-W.; Hsu, K.-H.; Young, T.-H. Direct-write laser micromachining and universal surface modi_cation of PMMA for device development. Sens. Actuators B Chem 2004, 99, 186_196.

(23) Ross, D.; Johnson, T. J.; Locascio, L. E. Imaging of Electroosmotic Flow in Plastic Microchannels. Anal. Chem. 2001, 73, 2509_2515.

(24) Milanova, D.; Chambers, R. D.; Bahga, S. S.; Santiago, J. G. E_ect of PVP on the electroosmotic mobility of wet-etched glass microchannels. Electrophoresis 2012, 33, 3259_3262.

(25) Kaneta, T.; Ueda, T.; Hata, K.; Imasaka, T. Suppression of electroosmotic_ow and its application to determination of electrophoretic mobilities ina a poly(vinylpyrrolidone)-coated capillary. J. of Chromatogr. A. 2006, 1106, 52_55.

(26) Gao, Q.; Yeung, E. S. A Matrix for DNA Separation: Genotyping and Sequencing Using Poly(vinylpyrrolidone) Solution in Uncoated Capillaries. Anal. Chem. 1998, 70, 1382_1388.

(27) Busher, J. T. In Serum ALbumin and Globulin, 3rd ed.; K., W. H., D., H. W., J. W., H., Eds.; Boston: Butterworths, 1990; Chapter 101, pp 497_499.

(28) lvarsson, M.; Carlson, J. In Extraction, Quantitation, and Evaluation of Function DNA from Various Sample Types ; Dillner, J., Ed.; Springer, 2011; Vol. 675; Chapter 14, pp 261_277.

(29) Piruska, A.; Nikcevic, I.; Lee, S. H.; Ahn, C.; Heineman, W. R.; Limbach, P. A.; Seliskar, C. J. The auto_uorescence of platic materials and chips measured under laser irradiation. Lab Chip 2005, 5, 1348_1354.

(30) Montes, R. J.; Butler, J. E.; Ladd, A. J. Trapping DNA with a high throughput mi-cro_uidic device. Electrophoresis 2019, 40, 437_446.

(31) Nguyen, N.-T.; Wereley, S. Fundamentals and Applications of Micro_uidics, 2nd ed.; Artech Print on Demand, 2006; Chapter 2, pp 20_30.

1. A microfluidic device system for isolating or detecting nucleic acids, comprising
a microcapillary having a first end and a second end with a length (L) longer than a width (W), and wherein one or more inner surfaces of the microcapillary are coated with a coating;
a fluid inlet at the first end fluidically connected to the microcapillary;
a fluid outlet at the second end fluidically connected to the microcapillary; and
a buffer.

2. The system of claim 1, wherein the first end, the second end, or both are greater in cross-sectional area than the section of microcapillary between the first and second ends.

3.-7. (canceled)

8. The system of claim 1, wherein the coating is poly(N,N-dimethacrylamide) (PDMAC) or poly(diallyldimethylammonium) chloride (PDADMAC).

9. The system of claim 1, wherein the system comprises one or more of polymethylmethacrylate (PMMA), polydimethylsiloxane (PDMS), polycarbonate, polystyrene, polyethylene, or glass.

10. (canceled)

11. The system of claim 1, further comprising a sample injection port fluidically connected to the microcapillary and positioned in between the fluid inlet and fluid outlet on the first end of the microcapillary.

12.-14. (canceled)

15. The system of claim 1, further comprising an electric current generator configured to generate an electrophoretic flow through the microcapillary.

16. The system of claim 1, further comprising an inlet tank fluidically connected to the fluid inlet and an outlet tank fluidically connected to the fluid outlet.

17. (canceled)

18. The system of claim 1, further comprising a syringe pump in fluidic communication with the fluid outlet.

19.-20. (canceled)

21. The system of claim 1, further comprising a pump to drive fluid motion through the system.

22.-24. (canceled)

25. The system of claim 1, further comprising a micrograph image collecting apparatus.

26.-27. (canceled)

28. A method for isolating or detecting nucleic acids, comprising providing the system of claim 1;
providing a sample comprising nucleic acids to the sample inlet or sample injection port of the microcapillary;
generating a fluid flow from the fluid inlet to the fluid outlet with a centerline velocity v₀ along a longitudinal axis of the microcapillary; and
providing an electric current to the microcapillary, wherein the electric current is configured to generate an electrophoretic velocity v e that is directionally opposed to the fluid flow and centerline velocity v; and
detecting or isolating nucleic acids at a stagnation region of interest near the fluid inlet.

29. The method of claim 28, further comprising illuminating the system with light from a light source for a period of time prior to providing the sample.

30-31. (canceled)

32. The method of claim 28, wherein a ratio of centerline velocity v e to electrophoretic velocity vₑ is about 20 to about 200.

33. The method of claim 32, wherein the centerline velocity v₀ is about 4 mm/s to about 12 mm/s.

34. The method of claim 32, wherein the electrophoretic velocity v e is about −0.03 mm/s to about −0.15 mm/s or about +0.03 mm/s to about +0.15 mm/s

35. The method of claim 28, wherein the nucleic acids comprise nucleic acids with a λ_{D} greater than a diameter of a backbone of the nucleic acids.

36. The method of claim 35, wherein the diameter of the backbone is about 2 nm.

37. The method of claim 28, wherein the nucleic acids comprise at least one nucleic acid with a length of at least 30 to 100 bases with a sequence having at least 95% sequence homology or greater with a viral RNA sequence of SARS-CoV-2.

38. The method of claim 28, wherein the nucleic acids comprise at least one nucleic acid with a length of at least 30 to 100 bases with a sequence having at least 95% sequence homology or greater with a viral RNA sequence of NCO Reference Sequence NC_045512.2.

39. The method of claim 28, wherein detecting or isolating nucleic acids at the stagnation region of interest near the fluid inlet comprises isolating DNA and RNA and detecting only RNA or only DNA.
